(19) United States
(12) Patent Application Publication
(10) Pub. No.: US 2024/0329064 A1
(43) Pub. Date: Oct. 3, 2024

(54) METHOD FOR DETECTING DRUG ANTIBODY IN SPECIMEN

(71) Applicant: Shenzhen Blood Center (Shenzhen Institute of Transfusion Medicine), Shenzhen City (CN)

(72) Inventors: Yanlian LIANG, Shenzhen City (CN); Xiongchi TANG, Guangxi (CN); Shuang LIANG, Shenzhen City (CN); Yunping XU, Shenzhen City (CN); Fuling ZHONG, Shenzhen City (CN)

(21) Appl. No.: 18/376,678

(22) Filed: Oct. 4, 2023

(30) Foreign Application Priority Data
Mar. 30, 2023 (CN) 2023103633289

(51) Int. Cl. G01N 33/94; G01N 33/68

(52) U.S. Cl. CPC G01N 33/94; G01N 33/6854; G01N 2496/05

(57) ABSTRACT

Provided is a method for detecting a drug antibody in a specimen. In the present disclosure, the drug antibody is detected by flow cytometry for the first time. During the detection, self cells of a patient are used as indicator cells. Moreover, nine systems are set up, including: a blank system, a negative control system, a positive control system, a self-control system, a drug-sensitized self-cell control system, a drug-antibody reaction detection system, an enzyme-sensitized self-control system, an enzyme-reaction drug detection system, and an enzyme-reaction drug control system. The method avoids interference from autoantibodies and irregular antibodies of red blood cells, and also avoids a false positive result of detection caused by combination of enzymes or drugs with self red blood cells. The method for detecting a drug antibody can simultaneously detect an affinity of the drug antibody to red blood cells and identify a type of the drug antibody.

### CROSS REFERENCE TO RELATED APPLICATION

This patent application claims the benefit and priority of Chinese Patent Application No. 2023103633289, filed with the China National Intellectual Property Administration on Mar. 30, 2023, the disclosure of which is incorporated by reference herein in its entirety as part of the present application.

### TECHNICAL FIELD

The present disclosure belongs to the technical field of antibody detection, and in particular relates to a method for detecting a drug antibody in a specimen.

### BACKGROUND

The immunogenicity of a drug refers to an ability of the drug to induce an immune response or immune-related events against itself or related proteins. Immunogenicity has wide-ranging implications. This mechanism may have no effect, or may affect an efficacy of the drug, and may even cause serious side effects in more serious cases. When an immune response occurs, the drug acts as an antigen and stimulates the body to produce drug antibodies. The drug antibodies can seriously affect the efficacy of the drug, or destroy (kill) blood cells in the human body. In severe cases, the drug antibodies may lead to hemolytic anemia, or result in ineffective blood transfusion and even life-threatening.

For detection methods of the drug antibody, it is generally necessary to pretreat a specimen to be tested using high-density enrichment magnetic beads. This pretreatment realizes the detection of a total anti-drug antibody (ADA) in a form including a drug-ADA complex in the specimen, thereby overcoming the influence of high-concentration drug proteins in the specimen and improving the drug resistance of an ADA detection method. However, the pretreatment has complicated scheme and high cost. During the detection by enzyme-linked immunosorbent assay (ELISA), after a general drug antigen is combined with an antibody, colorimetric determination is conducted, and a concentration is calculated by a curve equation. However, the method has poor sensitivity. The detection can also be conducted by a microcolumn gel method. In this method, the drug antigen is combined with the antibody, and obtained results are observed through a gel column. However, the method still has subjective determination differences and uncontrollable factors of sensitivity. Moreover, the method uses self-selected allogeneic cells as indicator cells, and cannot avoid interference from autoantibodies and irregular antibodies of red blood cells.

Accordingly, there is an urgent need for a drug antibody detection method with simple protocols, accurate results, and shielding from various interferences.

### SUMMARY

A purpose of the present disclosure is to provide a method for detecting a drug antibody in a specimen. The method can avoid interference from alloantibodies (that is, irregular antibodies of red blood cells) and autoantibodies in the specimen, and realize simple, efficient, and accurate detection. Moreover, the method can complete detection of an affinity of the drug antibody to red blood cells and identification of a type of the drug antibody.

The present disclosure provides a method for detecting a drug antibody in a specimen, including the following steps: preparing at least nine reaction systems; conducting flow cytometry on the at least nine reaction systems sequentially; and determining presence or absence of the drug antibody, a type of the drug antibody, and an affinity of the drug antibody to red blood cells according to results of the flow cytometry; where

the nine reaction systems include a blank system, a negative control system, a positive control system, a self-control system, a drug-sensitized self-cell control system, a drug-antibody reaction detection system, an enzyme-sensitized self-control system, an enzyme-reaction drug detection system, and an enzyme-reaction drug control system;

the blank system includes self cells and phosphate-buffered saline (PBS) (1×, pH=7.4, the same below);

the negative control system includes RhD negative cells, IgG anti-D serum, and the PBS;

the positive control system includes RhD positive cells, the IgG anti-D serum, and the PBS;

the self-control system includes the self cells, the PBS, and self plasma;

the drug-sensitized self-cell control system includes the self cells, the PBS, and a drug liquid;

the drug-antibody reaction detection system includes drug-sensitized self cells, the drug liquid, and the self plasma;

the enzyme-sensitized self-control system includes enzyme-sensitized self cells, the PBS, and the self plasma;

the enzyme-reaction drug detection system includes the enzyme-sensitized self cells, the drug liquid, and the self plasma;

the enzyme-reaction drug control system includes the enzyme-sensitized self cells, the drug liquid, and the PBS; and

the drug liquid is a drug solution that produces a drug antibody to be tested.

Preferably, the self plasma is derived from a serum specimen or a plasma specimen of a patient with a medication history.

Preferably, the self plasma is a supernatant obtained after conducting centrifugation on the serum specimen or the plasma specimen of the patient with the medication history; and the centrifugation is conducted at 10,000 rpm for 15 min to 20 min.

Preferably, the self cells are self red blood cells of the patient with the medication history.

Preferably, a preparation method of the drug-sensitized self cells includes: mixing a normal saline instead of partial plasma with self cells of a patient and a drug at a medication concentration of the human body, and conducting incubation to obtain the drug-sensitized self cells.

Preferably, a preparation method of the enzyme-sensitized self cells includes: mixing self cells of a patient with a papain solution at an equal volume, and conducting incubation to obtain the enzyme-sensitized self cells.

Preferably, the self cells, the drug-sensitized self cells, and the enzyme-sensitized self cells each are configured into a 5% suspension during preparation of respective systems.

Preferably, each 150 μL of the blank system includes 50 μL of the self cells (5%) and 100 μL of the PBS;

each 300 μL of the negative control system includes 100 μL of the RhD negative cells (5%), 100 μL of the IgG anti-D serum, and 100 μL of the PBS;

each 300 μL of the positive control system includes 100 μL of the RhD positive cells (5%), 100 μL of the IgG anti-D serum, and 100 μL of the PBS;

each 300 μL of the self-control system includes 100 μL of the self cells (5%), 100 μL of the PBS, and 100 μL of the self plasma;

each 300 μL of the drug-sensitized self-cell control system includes 100 μL of the self cells (5%), 100 μL of the PBS, and 100 μL of the drug liquid;

each 300 μL of the drug-antibody reaction detection system includes 100 μL of the drug-sensitized self cells (5%), 100 μL of the drug liquid, and 100 μL of the self plasma;

each 300 μL of the enzyme-sensitized self-control system includes 100 μL of the enzyme-sensitized self cells (5%), 100 μL of the PBS, and 100 μL of the self plasma;

each 300 μL of the enzyme-reaction drug detection system includes 100 μL of the enzyme-sensitized self cells (5%), 100 μL of the drug liquid, and 100 μL of the self plasma; and

each 300 μL of the enzyme-reaction drug control system includes 100 μL of the enzyme-sensitized self cells (5%), 100 μL of the drug liquid, and 100 μL of the PBS.

Preferably, the method further includes the following steps before the flow cytometry is conducted: conducting incubation on each of the reaction systems at 37° C., mixing with a fluorescently-labeled antibody diluent obtained by conducting dilution on 500 μL of the PBS and 5 μL of a fluorescent stock solution at 1:(50-100), and conducting incubation in the dark.

Preferably, a type of an induction drug of the drug antibody includes a drug-sensitized type, a drug-added type, and an enzyme-treated drug-antibody immune complex type.

Beneficial effects: in the present disclosure, the drug antibody is detected by flow cytometry for the first time. During the detection, self cells of a patient are used as indicator cells. Moreover, three systems are set up, including a self-cell control, an enzyme-sensitized control, and a drug-sensitized control. The method avoids interference from autoantibodies and irregular antibodies of red blood cells, and also avoids a false positive result of detection caused by combination of enzymes or drugs with self red blood cells.

The method for detecting a drug antibody can simultaneously detect an affinity of the drug antibody to red blood cells and identify a type of the drug antibody. The affinity refers to an ability of the drug or the drug antibody or a drug-drug antibody complex to bind to red blood cells, and can also be called a strength of adhesion to a surface of the red blood cell. The affinity is reflected by a reading of a fluorescence intensity (namely a Mean value). If the fluorescence is strong, the affinity is strong, and vice versa. If the Mean value of a positive control or a test tube is greater than the Mean value of a negative control, the specimen to be tested is positive (namely successful combination of antigen-antibody). The experiment has controllable process and credible results. All detection systems are compared with the Mean of a negative control system. If the Mean value of the detection system is greater than the Mean value of the negative control, the specimen to be tested is determined as positive. If the mean value of the drug-antibody reaction detection system is greater than the mean values of the self-control system and the drug-sensitized self-cell control system, and the mean value of the enzyme-reaction drug detection system is greater than the mean values of the enzyme-sensitized self-control system and the enzyme-reaction drug control system, the specimen to be tested is determined to be positive for the drug antibody. The method has strong sensitivity and intuitive data readout, thereby improving a detection rate of weak antibodies.

### BRIEF DESCRIPTION OF THE DRAWINGS

To describe the technical solutions in the examples of the present disclosure or in the prior art more clearly, the accompanying drawings required for the examples will be briefly described below.

FIG. 1 shows a specimen addition pattern of the systems;

FIGS. 2A-I show flow cytometry diagrams of the nine specimen injection systems;

FIGS. 3A-C show flow cytometry results of Tubes 0 to 2 in Example 1;

FIGS. 4A-C show flow cytometry results of Tubes 3 to 5 in Example 1;

FIGS. 5A-C show flow cytometry results of Tubes 6 to 8 in Example 1;

FIGS. 6A-C show flow cytometry results of Tubes 1, 2, and 5 in Example 2;

FIGS. 7A-C show flow cytometry results of Tubes 1, 2, and 5 in Example 3;

FIGS. 8A-C show flow cytometry results of Tubes 1, 2, and 5 in Example 4;

FIGS. 9A-D show flow cytometry results of Tubes 1, 2, 5, and 7 in Example 5; and

FIGS. 10A-C show flow cytometry results of Tubes 1, 2, and 5 in Example 6.

### DETAILED DESCRIPTION OF THE EMBODIMENTS

The present disclosure provides a method for detecting a drug antibody in a specimen, including the following steps: preparing at least nine reaction systems; conducting flow cytometry on the at least nine reaction systems sequentially; and determining presence or absence of the drug antibody, a type of the drug antibody, and an affinity of the drug antibody to red blood cells according to results of the flow cytometry; where

the nine reaction systems include a blank system, a negative control system, a positive control system, a self-control system, a drug-sensitized self-cell control system, a drug-antibody reaction detection system, an enzyme-sensitized self-control system, an enzyme-reaction drug detection system, and an enzyme-reaction drug control system;

the blank system includes self cells and PBS;

the negative control system includes RhD negative cells, IgG anti-D serum, and the PBS;

the positive control system includes RhD positive cells, the IgG anti-D serum, and the PBS;

the self-control system includes the self cells, the PBS, and self plasma;

the drug-sensitized self-cell control system includes the self cells, the PBS, and a drug liquid;

the drug-antibody reaction detection system includes drug-sensitized self cells, the drug liquid, and the self plasma;

the enzyme-sensitized self-control system includes enzyme-sensitized self cells, the PBS, and the self plasma;

the enzyme-reaction drug detection system includes the enzyme-sensitized self cells, the drug liquid, and the self plasma;

the enzyme-reaction drug control system includes the enzyme-sensitized self cells, the drug liquid, and the PBS; and

the drug liquid is a drug solution that produces a drug antibody to be tested.

In the present disclosure, the self plasma is preferably derived from a serum specimen or a plasma specimen of a patient with a medication history. In an example, the serum specimen or the plasma specimen of the patient with the medication history is preferably centrifuged at 10,000 rpm for 15 min to 20 min, and an obtained supernatant is collected to obtain the self plasma.

In the present disclosure, the self cells are preferably patient's self red blood cells. When a system is prepared, the patient's self red blood cells are washed with a normal saline, prepared into a 5% suspension, and the 5% suspension is used to prepare the system. The 5% suspension is preferably using 1×PBS, pH=7.4 as a diluent, and adding 50 μL of packed red blood cells into 1,000 μL of the PBS, to obtain a suspension of the red blood cells with a concentration of 5%. The washing is preferably conducted 5 times. A preparation method of the drug-sensitized self cells includes preferably: mixing a normal saline instead of partial plasma with self red blood cells of a patient and a drug at a medication concentration of the human body, and conducting incubation to obtain the drug-sensitized self cells. Preferably, the normal saline is used to replace 55% (v/v) of the plasma, mixed with 45% (v/v) of the packed red blood cells and the drug, and then incubated at 37° C. for 1.5 h to obtain the 5% suspension, so as to prepare the system. A preparation method of the enzyme-sensitized self cells includes preferably: mixing self red blood cells of a patient with a papain solution at an equal volume, and conducting incubation to obtain the enzyme-sensitized self cells. The incubation is preferably conducted at 37° C. for 15 min, and an obtained product is washed 5 times with a large amount of normal saline to obtain the 5% suspension, so as to prepare the subsequent system.

In the present disclosure, since each of the systems are used for flow cytometry detection, the systems each are prepared in a centrifuge tube. Therefore, each of the systems may also be numbered, including:
Tube 0: 50 μL of the self cells (5%) and 100 μL of the PBS;
Tube 1: the negative control system includes 100 μL of the RhD negative cells (5%), 100 μL of the IgG anti-D serum, and 100 μL of the PBS;
Tube 2: the positive control system includes 100 μL of the RhD positive cells (5%), 100 μL of the IgG anti-D serum, and 100 μL of the PBS;
Tube 3: the self-control system includes 100 μL of the self cells (5%), 100 μL of the PBS, and 100 μL of the self plasma;
Tube 4: the drug-sensitized self-cell control system includes 100 μL of the self cells (5%), 100 μL of the PBS, and 100 μL of the drug liquid;
Tube 5: the drug-antibody reaction detection system includes 100 μL of the drug-sensitized self cells (5%), 100 μL of the drug liquid, and 100 μL of the self plasma;
Tube 6: the enzyme-sensitized self-control system includes 100 μL of the enzyme-sensitized self cells (5%), 100 μL of the PBS, and 100 μL of the self plasma;
Tube 7: the enzyme-reaction drug detection system includes 100 μL of the enzyme-sensitized self cells (5%), 100 μL of the drug liquid, and 100 μL of the self plasma; and
Tube 8: the enzyme-reaction drug control system includes 100 μL of the enzyme-sensitized self cells (5%), 100 μL of the drug liquid, and 100 μL of the PBS.

In the present disclosure, after Tubes 0 to 8 are prepared to obtain the systems, respectively, the method further includes preferably: after conducting incubation at 37° C., adding a fluorescently-labeled antibody diluent obtained by conducting dilution on 500 μL of the PBS and 5 μL of a fluorescent stock solution at 1:(50-100), mixing, conducting incubation in the dark, followed by determination on a machine. More preferably, after adding specimens into each of the tubes, the specimens each are incubated at 37° C. for 1.5 h, washed 3 times with PBS, and added with 100 μL of the fluorescently-labeled antibody diluent (the fluorescent antibodies are preferably added with FITC-labeled goat anti-human IgG Fc, and diluted 50 to 100 times before adding the specimens), incubated in the dark at 37° C. for 30 min±5 min, suspended in the PBS, placed in the dark at a room temperature, and determined within 2 h.

In the present disclosure, a procedure for on-board detection includes preferably: adjusting a voltage with Tube 0 (self cells), acquiring data after gating, and defaulting an optical channel voltage and a photoelectric magnification according to a current state of a flow cytometer used; loading a specimen into a test tube, acquiring 50 μL of the specimen, collecting 10,000 red blood cells within a set gate, and collecting a fluorescence intensity of a drug antigen-antibody conjugate on a surface of each red blood cell.

In the present disclosure, Tube 0 is used for “gating” during the flow cytometry detection. Tube 1 is a negative control tube for an antigen-antibody binding reaction. Tube 2 is a positive control tube for the antigen-antibody binding reaction. Tube 3 is a “zero” control tube for autoantibodies. Tube 4 is a “zero” control tube for drug-sensitized self cells. Tube 5 is a detection tube for drug antibody identification and cell affinity. Tube 6 is a “zero” detection tube of the enzyme-sensitized self cells, as a control. Tube 7 is an enzyme-dependent immunocomplex detection tube for drug antibody identification and cell affinity. Tube 8 is a “zero” control tube for enzyme- and drug-sensitized self cells.

By using the method of the present disclosure, the detection of the affinity of the drug antibody to red blood cells and the identification of the type of the drug antibody can be completed simultaneously. The affinity of the drug antibody to red blood cells is detected by the flow cytometry, so as to understand a destructive power of the drug antibody to red blood cells, that is, the lethality. This method is suitable for drug antibody identification of a drug-sensitized type (such as piperacillin, amoxicillin) and a drug-added type (such as ceftriaxone, vancomycin), as well as antibody detection of an enzyme-treated drug-antibody immune complex type. The method can avoid interference from alloantibodies (that is, irregular antibodies of red blood cells) and autoantibodies in the patient's plasma. The affinity refers to an ability of the drug or the drug antibody or a drug-drug antibody complex to bind to red blood cells, and can also be called a strength of adhesion to a surface of the red blood cell. The affinity is reflected by a reading of a fluorescence intensity (namely a Mean value). If the fluorescence is strong, the affinity is strong, and vice versa. The drug-drug antibody binds to red blood cells, sensitizes red blood cell membranes or changes a structure of the red blood cell membrane. Some antibodies can also stimulate complement to destroy red blood cells and cause red blood cell lysis, which is the so-called lethality. That is, the stronger binding capacity and more bound drug-drug antibody results in easier destruction of the red blood cells.

For drug-sensitized drugs (such as piperacillin and amoxicillin kits): when there are drug antibodies in the patient's plasma, when medication is conducted again, the drug can bind to the drug antibody when it enters the blood. However, the antibody in the plasma does not bind to the red blood cells after being combined with the drug, so it is necessary for the drug to sensitize the red blood cells first (since the drug-sensitized red blood cells may also cause fluorescence enhancement, in the present disclosure, a control tube (Tube 4) with drug sensitization and not being bound to antibody is set as a reference base for fluorescence readings). The plasma to be tested is added. If there is a corresponding drug antibody in the plasma, the drug antibody can combine with the red blood cells sensitized to the drug, and the fluorescence is enhanced.

For drug-added drugs (such as ceftriaxone and vancomycin kits): after the formation of immune complexes, the drugs free in plasma can bind to the surface of red blood cells after binding to antibodies. The enzyme reagents can enhance the ability of the drug-antibody complex to bind to red blood cells, and an enzyme-involved assay is suitable for the detection of immune complexes. Moreover, in the present disclosure, the enzyme-involved assay is set in each experiment, and is suitable for the detection of patients who take multiple drugs, so as not to miss a certain drug antibody.

In order to further illustrate the present disclosure, the method for detecting a drug antibody in a specimen provided in the present disclosure is described in detail below in conjunction with the accompanying drawings and examples, which are not to be construed as limiting the scope of protection of the present disclosure.

In the present disclosure, the specimens of each example are prepared through a same operation after collection:

Self plasma: the patient's serum or plasma specimen is centrifuged at 10,000 rpm for 15 min to 20 min, and a supernatant is collected for later use.

Patient's self cells: the cells are washed 5 times with a normal saline and prepared into a 5% suspension.

Drug-sensitized red blood cells: the patient's self cells are mixed with normal saline instead of plasma (55%)+packed red blood cells (45%)+drug (according to human drug concentration), mixed, incubated at 37° C. for 1.5 h, and prepared into a 5% suspension using PBS.

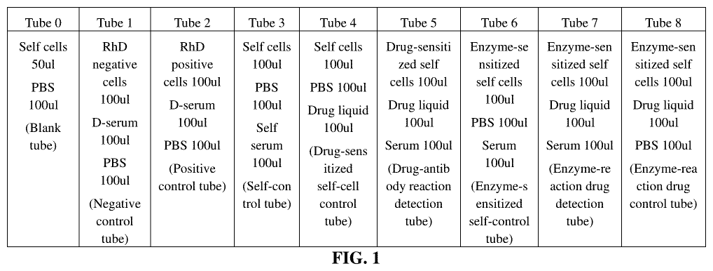

Enzyme-sensitized red blood cells: the patient's self cells are washed 5 times with a normal saline, mixed well with an equal volume of packed red blood cells+an equal volume of an enzyme solution, incubated at 37° C. for 15 min, washed 5 times with a large amount of the normal saline, and prepared into a 5% suspension with PBS. The sampling rules for each tube are shown in FIG. 1:
Tube 0: 50 μL of the self cells (5%) and 100 μL of the PBS;
Tube 1: the negative control system includes 100 μL of the RhD negative cells (5%), 100 μL of the IgG anti-D serum, and 100 μL of the PBS;
Tube 2: the positive control system includes 100 μL of the RhD positive cells (5%), 100 μL of the IgG anti-D serum, and 100 μL of the PBS;
Tube 3: the self-control system includes 100 μL of the self cells (5%), 100 μL of the PBS, and 100 μL of the self plasma;
Tube 4: the drug-sensitized self-cell control system includes 100 μL of the self cells (5%), 100 μL of the PBS, and 100 μL of the drug liquid;
Tube 5: the drug-antibody reaction detection system includes 100 μL of the drug-sensitized self cells (5%), 100 μL of the drug liquid, and 100 μL of the self plasma;
Tube 6: the enzyme-sensitized self-control system includes 100 μL of the enzyme-sensitized self cells (5%), 100 μL of the PBS, and 100 μL of the self plasma;
Tube 7: the enzyme-reaction drug detection system includes 100 μL of the enzyme-sensitized self cells (5%), 100 μL of the drug liquid, and 100 μL of the self plasma; and
Tube 8: the enzyme-reaction drug control system includes 100 μL of the enzyme-sensitized self cells (5%), 100 μL of the drug liquid, and 100 μL of the PBS.

After adding specimens according to the rules of different tubes, each obtained system is incubated at 37° C. for 1.5 h and washed 3 times with PBS. 100 μL of a diluted fluorescent antibody is added (the fluorescent antibody is preferably FITC-labeled goat anti-human IgG Fc according to the purpose of detection, and then diluted 50 to 100 times before adding the specimens), and incubated in the dark at 37° C. for 30 min±5 min. An obtained product is suspended in PBS, placed in the dark at a room temperature, and determined on a machine within 2 h. Tube 0 (self cells) is used to adjust a voltage and conduct gating to collect data, and an optical channel voltage and a photoelectric magnification are defaulted according to a current state of a flow cytometer used. A specimen is loaded into a test tube, 50 μL of the specimen is acquired, 10,000 red blood cells are collected within a set gate, and a fluorescence intensity of a drug antigen-antibody conjugate is collected on a surface of each red blood cell.

### Example 1

Wu, a patient at the Municipal People's Hospital, was tested for the drug antibody to ceftriaxone. The medication history of ceftriaxone was 4 d (2022.2.20-23), and the drug antibody to ceftriaxone was detected (by flow cytometry: Attune WXT).

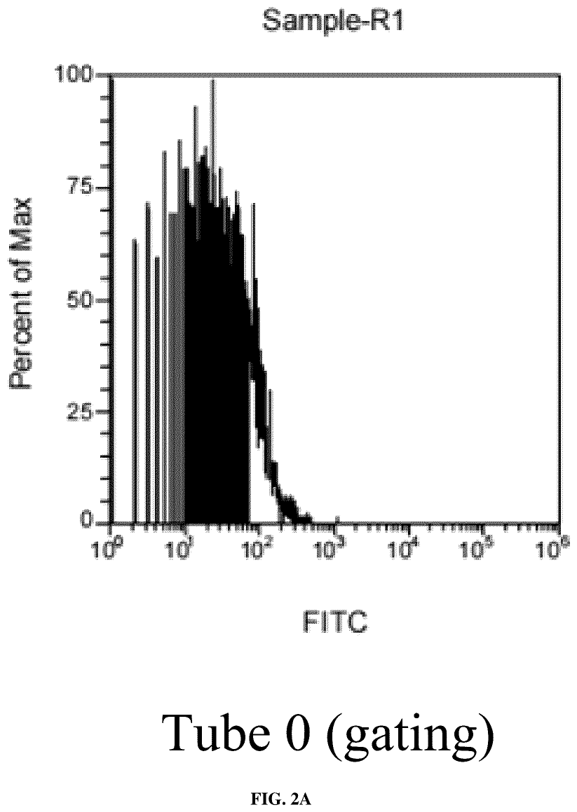

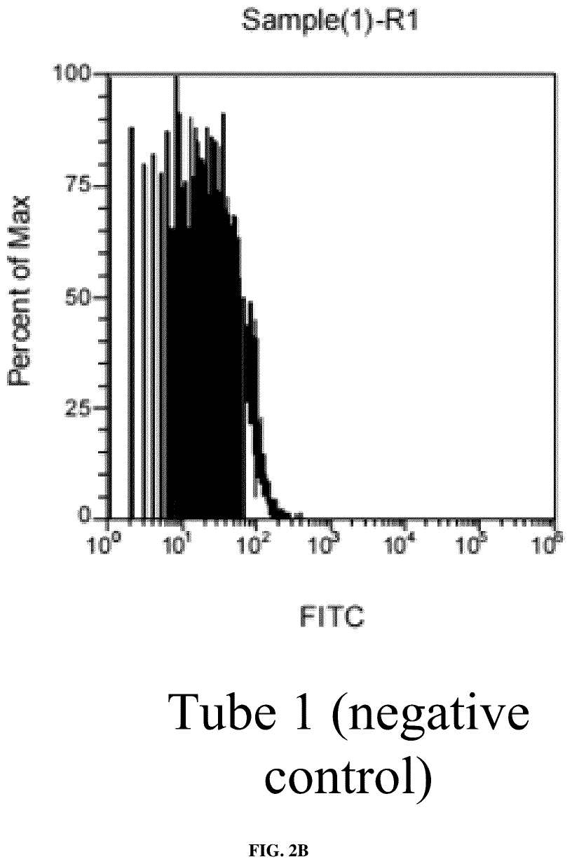

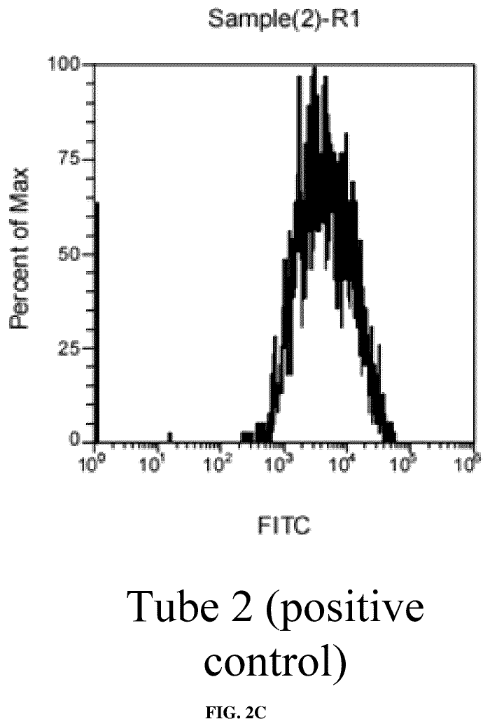

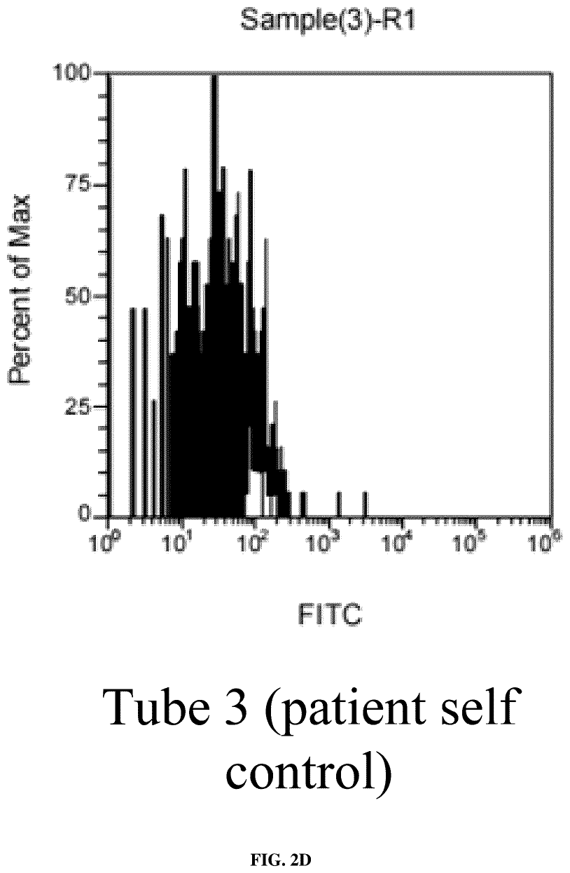

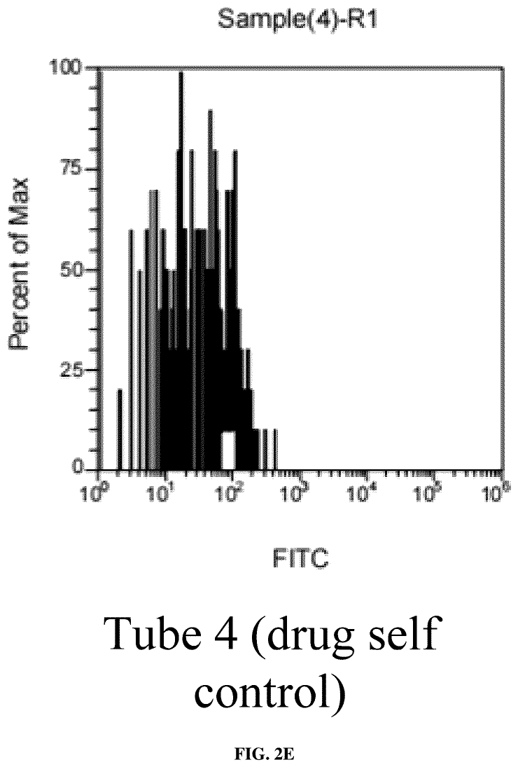

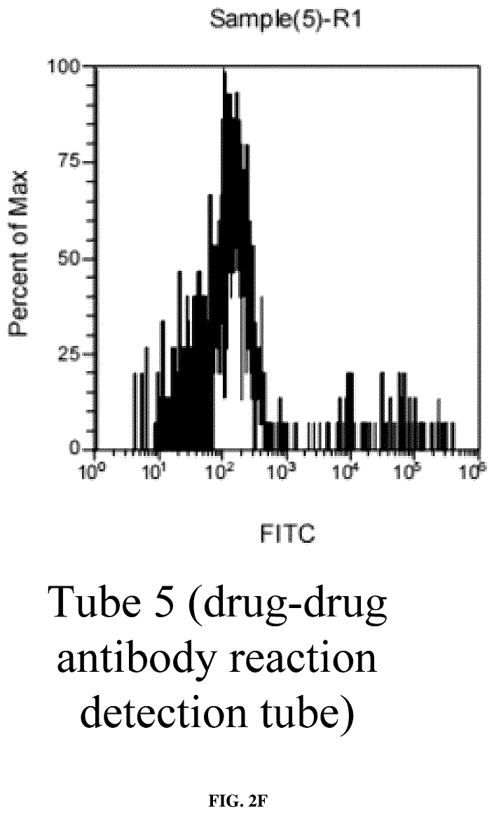

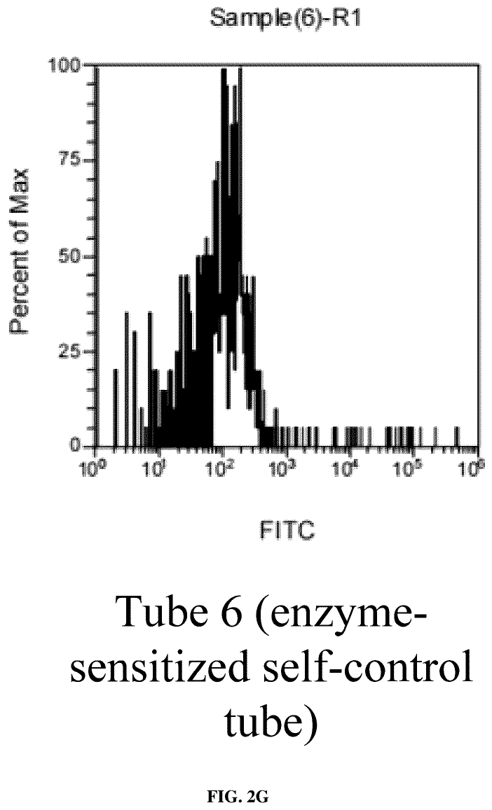

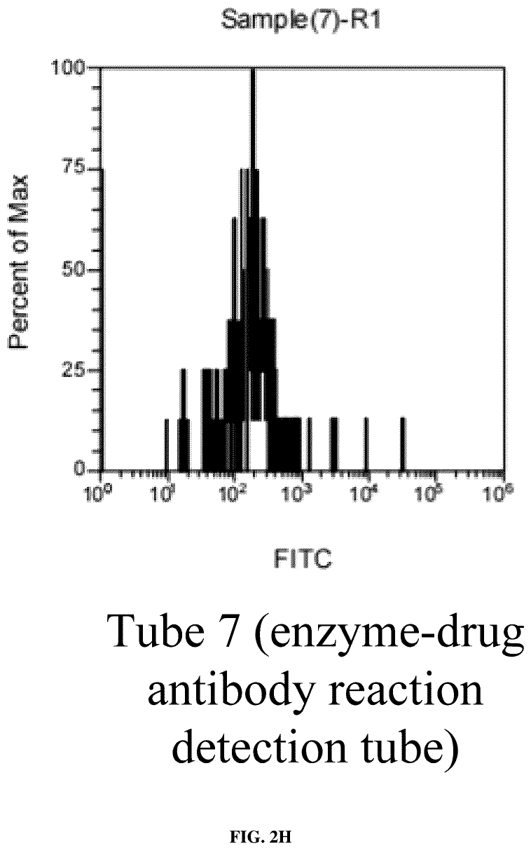

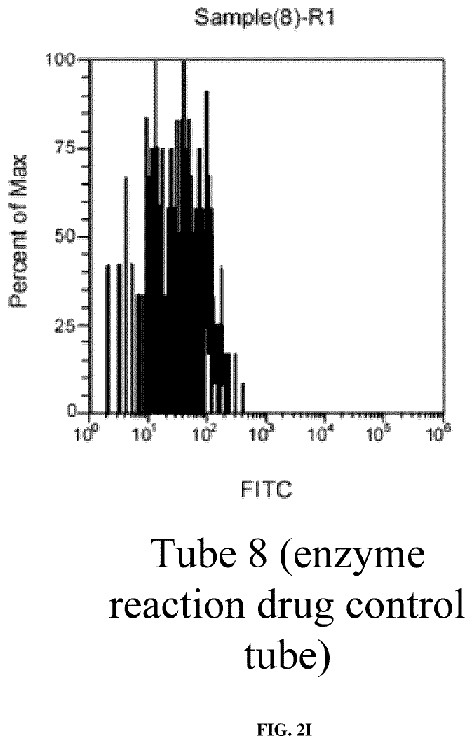

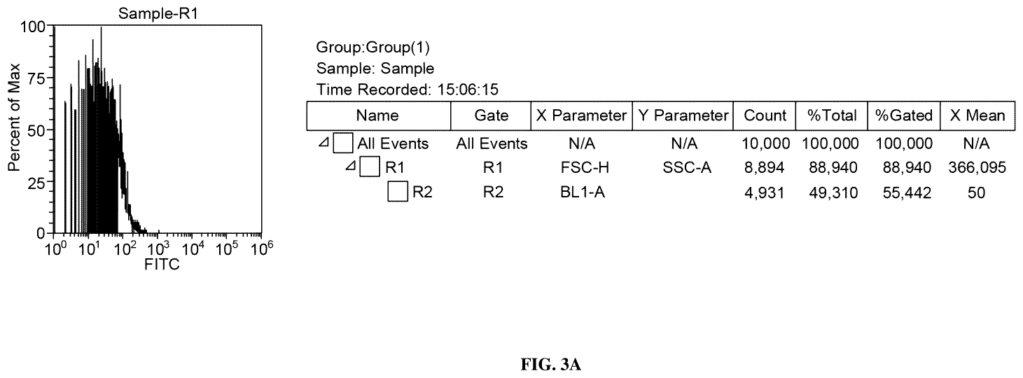

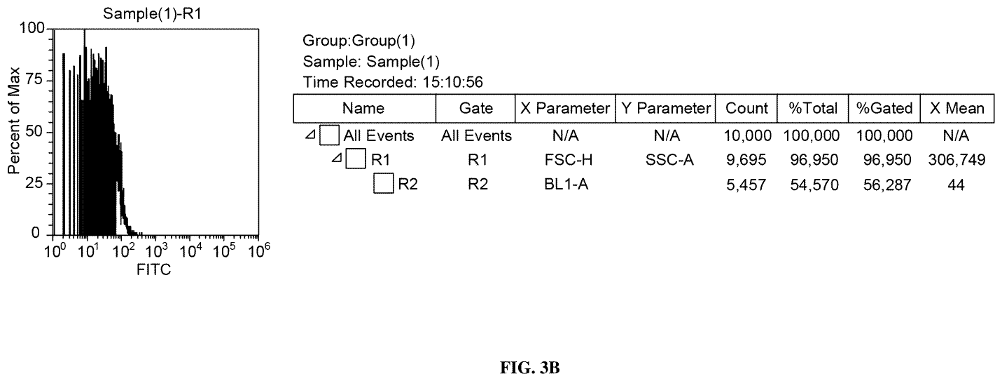

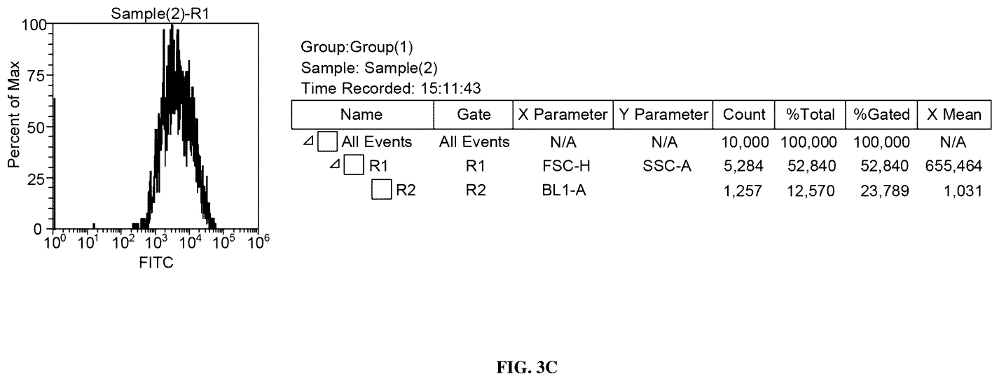

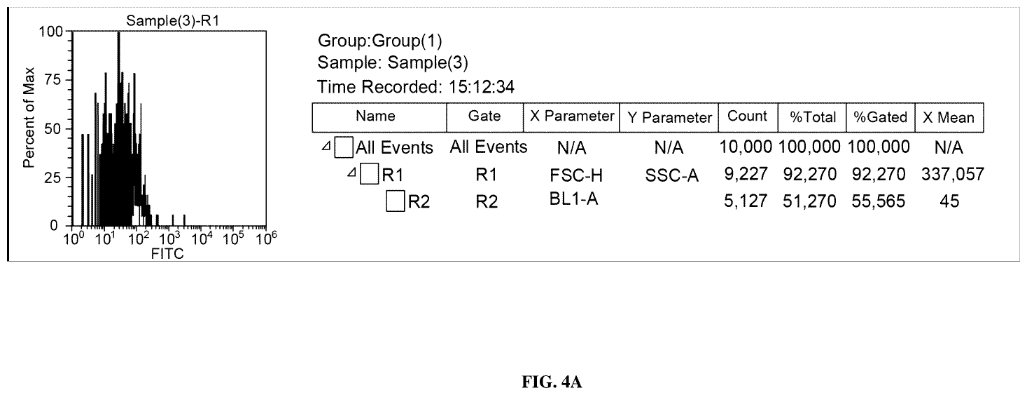

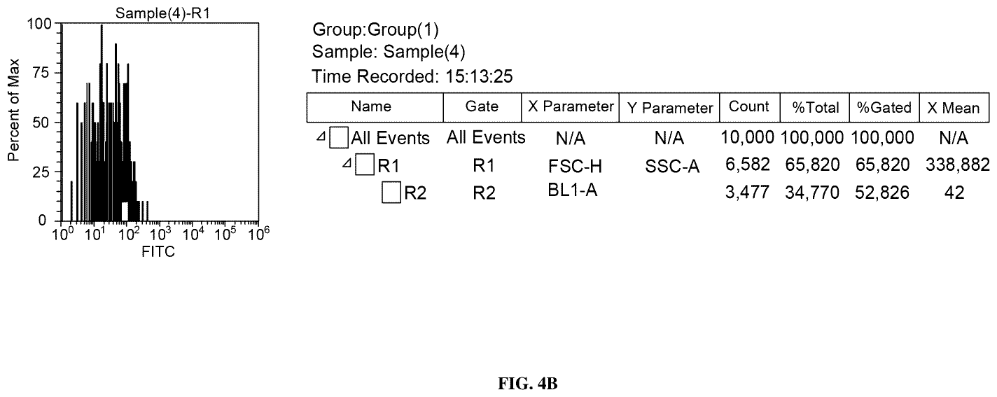

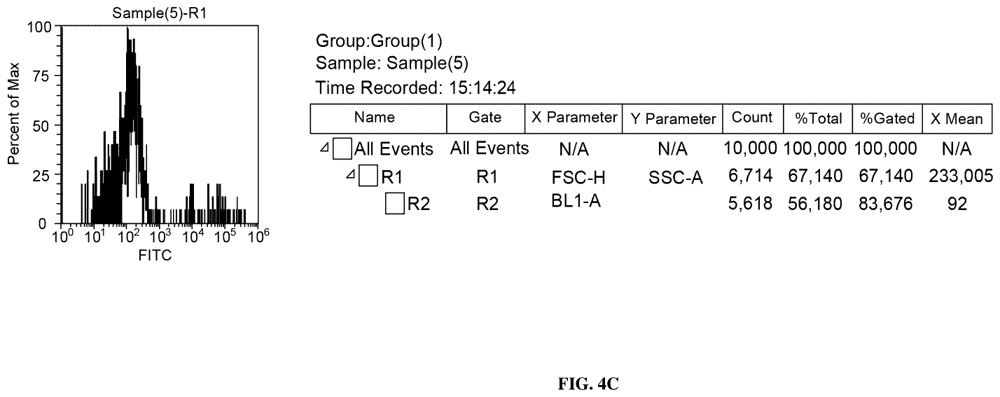

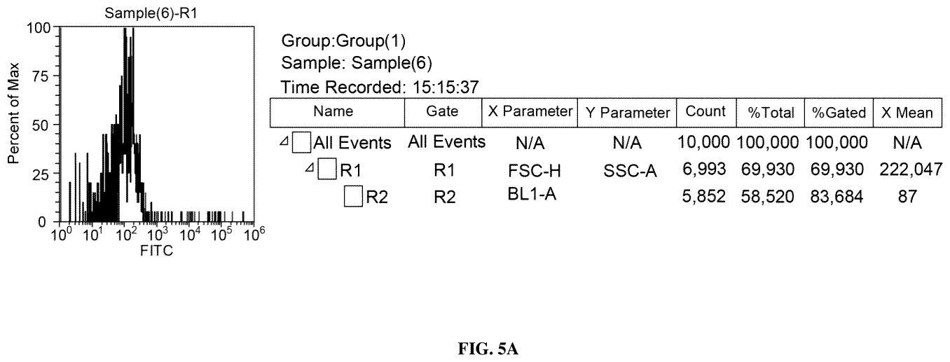

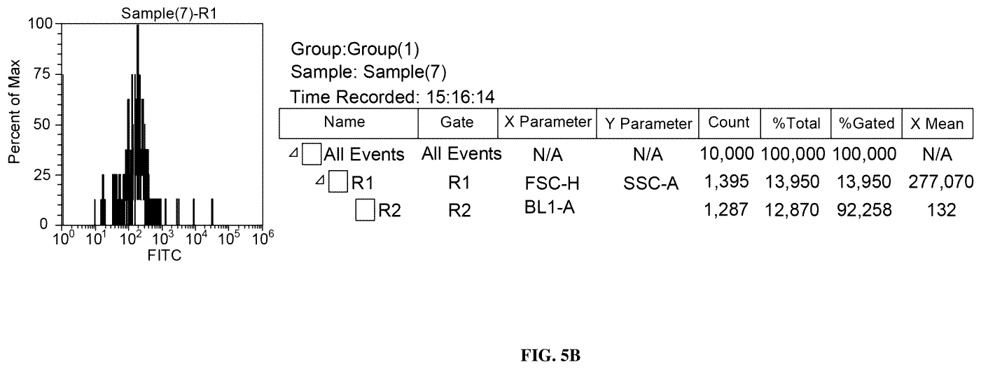

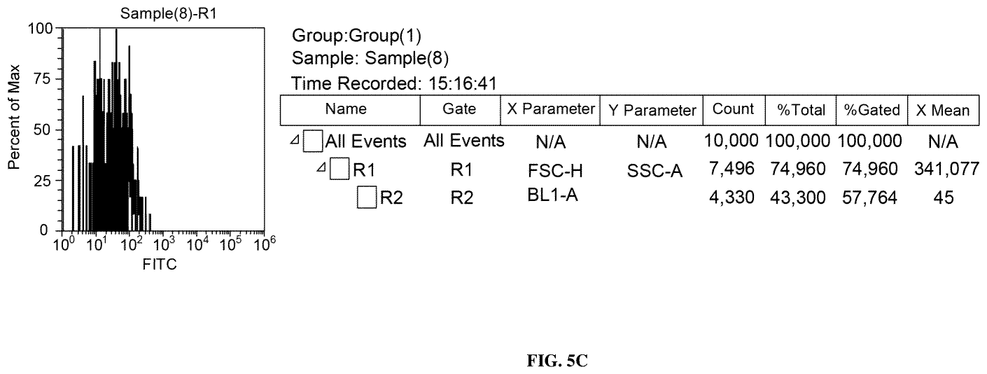

The results were shown in FIGS. 2A-I to FIGS. 5A-C and Table 1, the drug antibody to ceftriaxone was positive, and was a drug-added type.

TABLE 1
Mean value statistics and result determination of each system

| | Tube NO. | | | | | | | | |
|---|---|---|---|---|---|---|---|---|---|
| | Tube 0 | Tube 1 | Tube 2 | Tube 3 | Tube 4 | Tube 5 | Tube 6 | Tube 7 | Tube 8 |
| Mean | 50 | 44 | 1031 | 45 | 42 | 92 | 87 | 132 | 45 |
| value | | | | | | | | | |
| Results | | | | | | Positive | | Positive | |

### Example 2

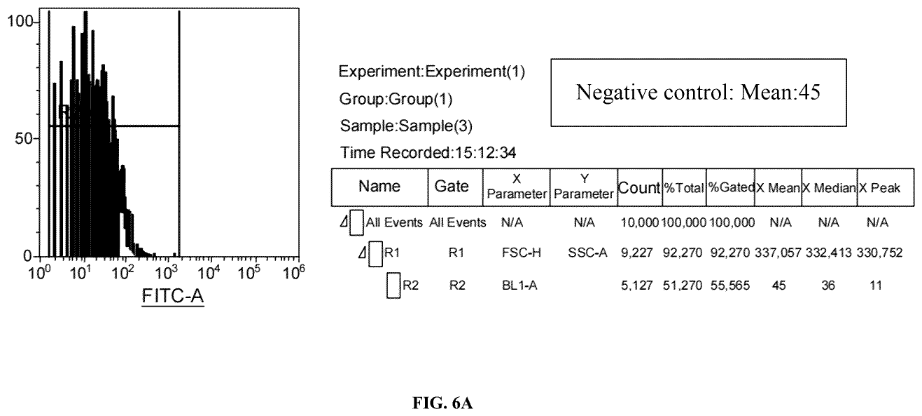

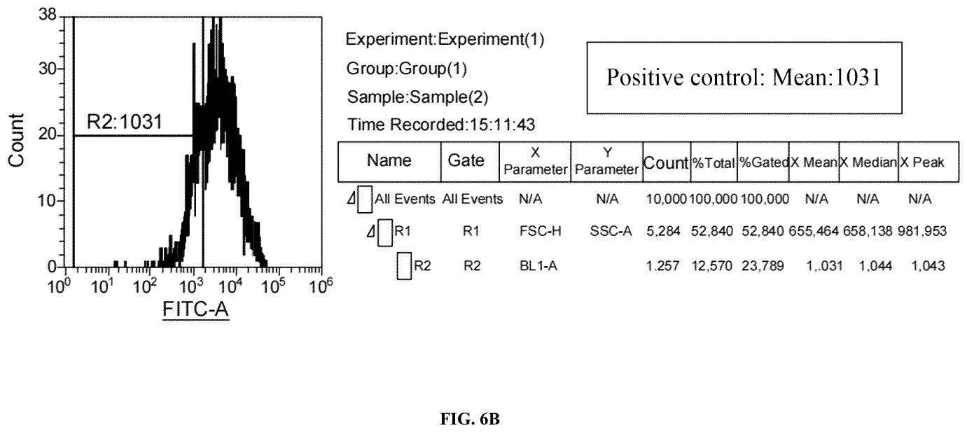

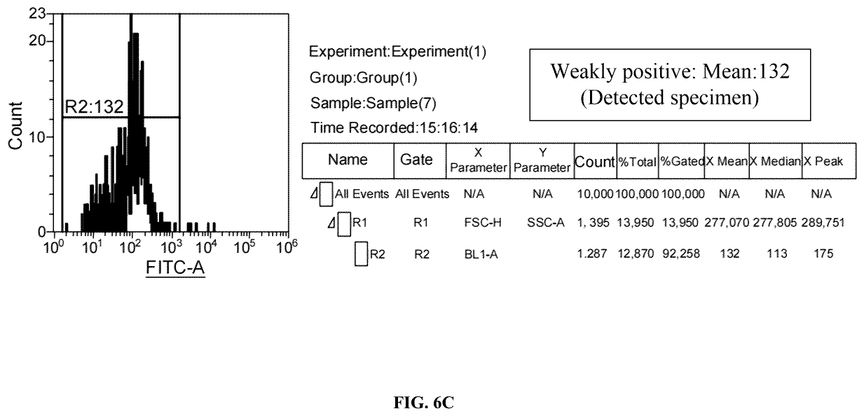

Jiao, a patient from the Third Hospital of the City, was tested on the 5th day of piperacillin administration in this medical history (2022.1.14-18), and the drug antibody to piperacillin was detected (by flow cytometer: Attune WXT). The results were shown in FIGS. 6A-C, the drug antibody to piperacillin was weakly positive.

### Example 3

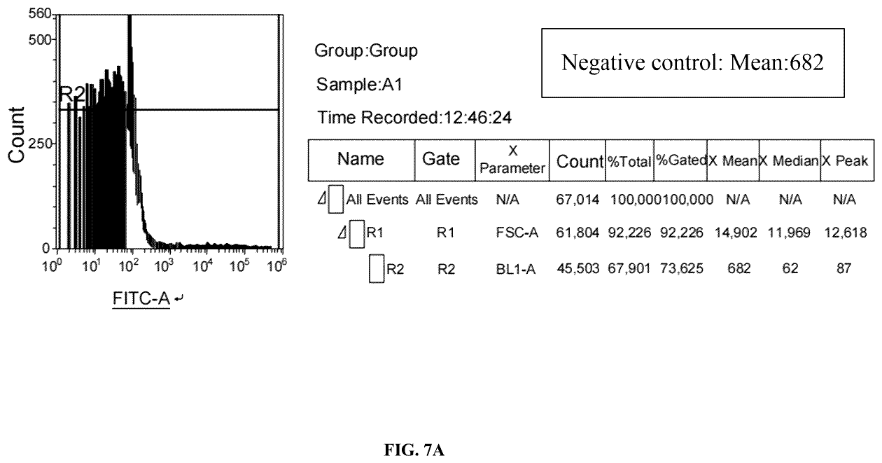

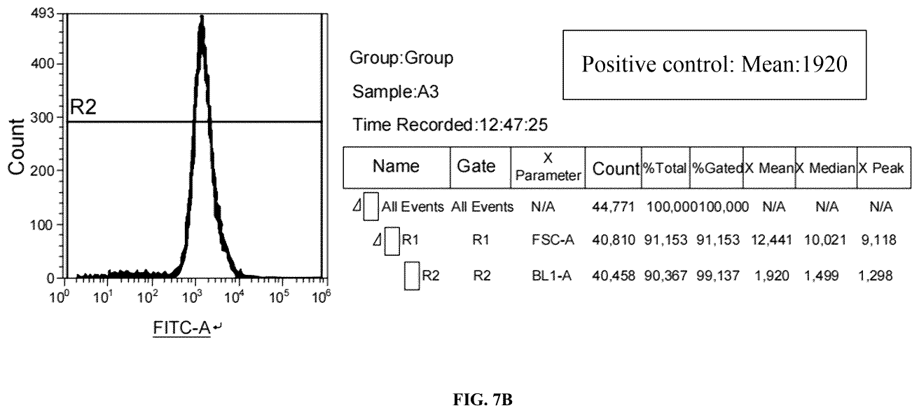

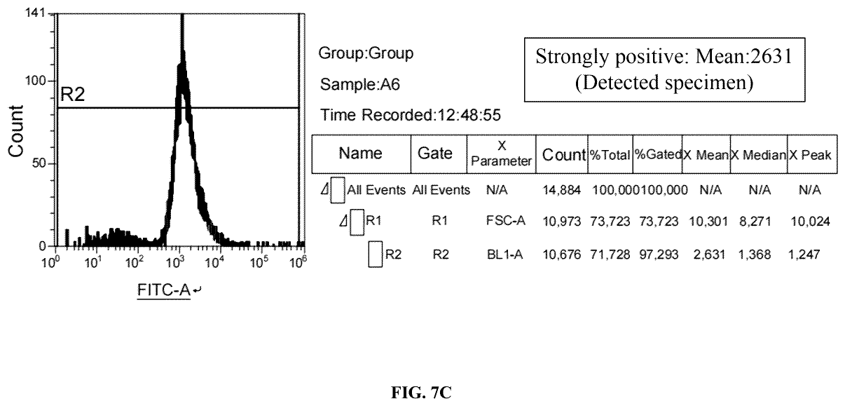

Qin, a patient from Children's Hospital, was tested on the 7th day of amoxicillin administration in this medical history (2021.11.10-16), and the drug antibody to amoxicillin was detected (by flow cytometer: BD FASCanto II). The results were shown in FIGS. 7A-C, the drug antibody to amoxicillin was strongly positive.

### Example 4

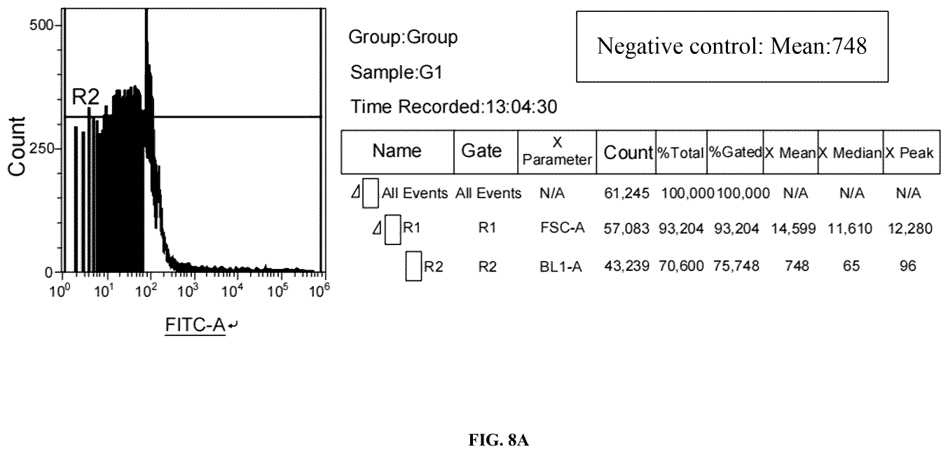

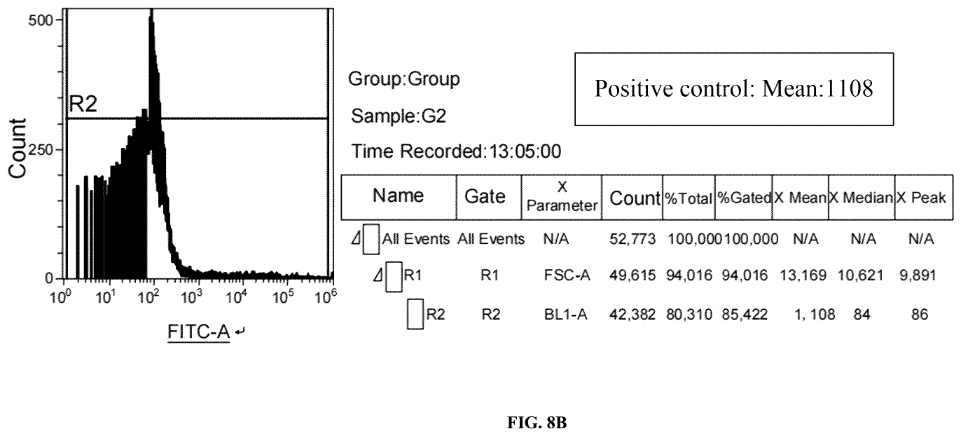

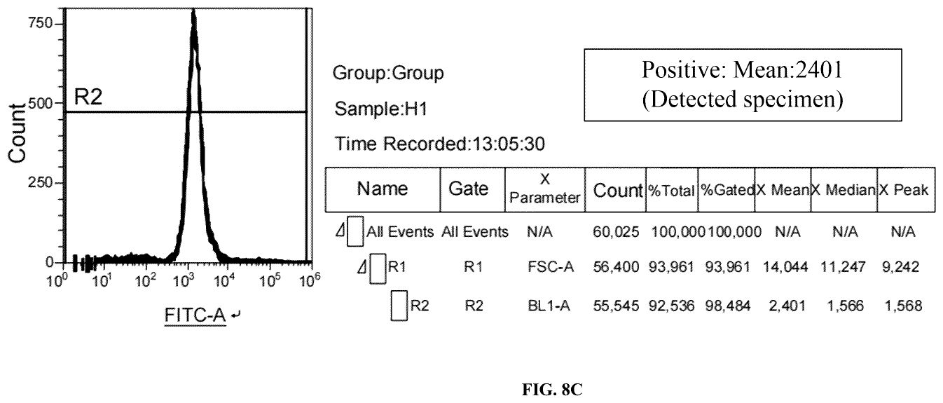

Peng, a patient from the Third Hospital of the City, was tested on the 6th day of piperacillin administration in this medical history (2021.11.14-19), and the drug antibody to piperacillin was detected (by flow cytometer: BD FASCanto II). The results were shown in FIGS. 8A-C, the drug antibody to piperacillin was positive.

### Example 5

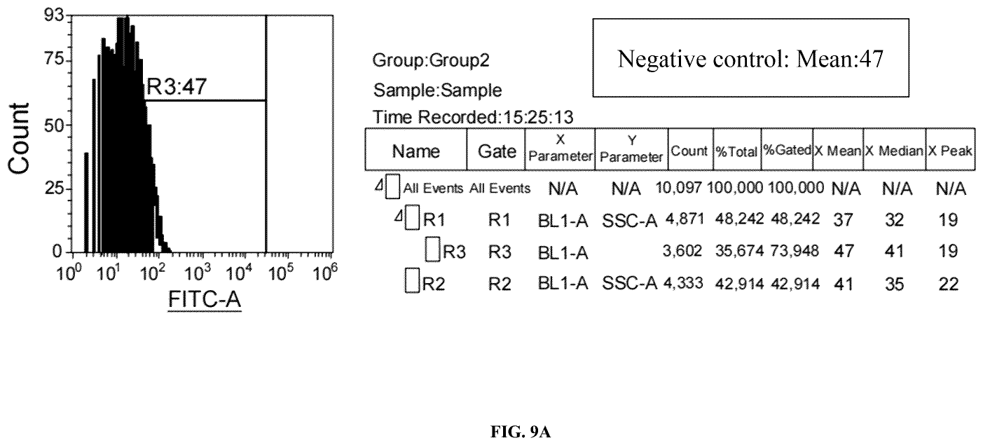

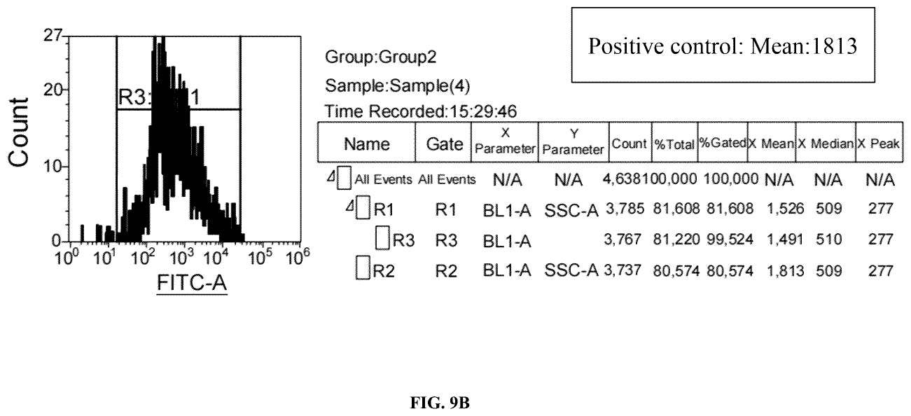

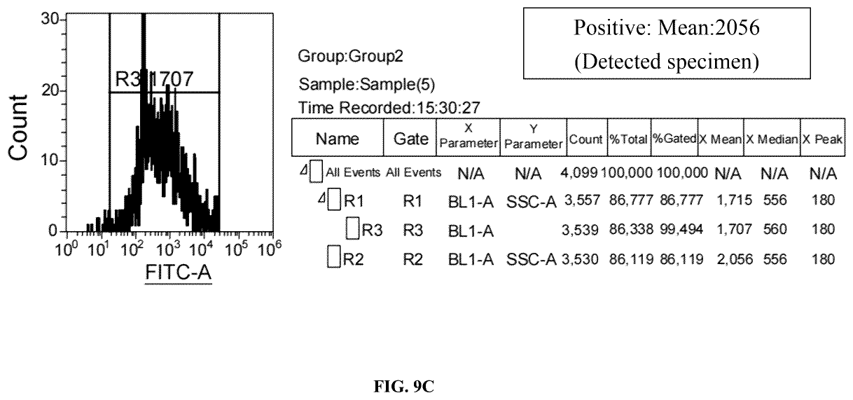

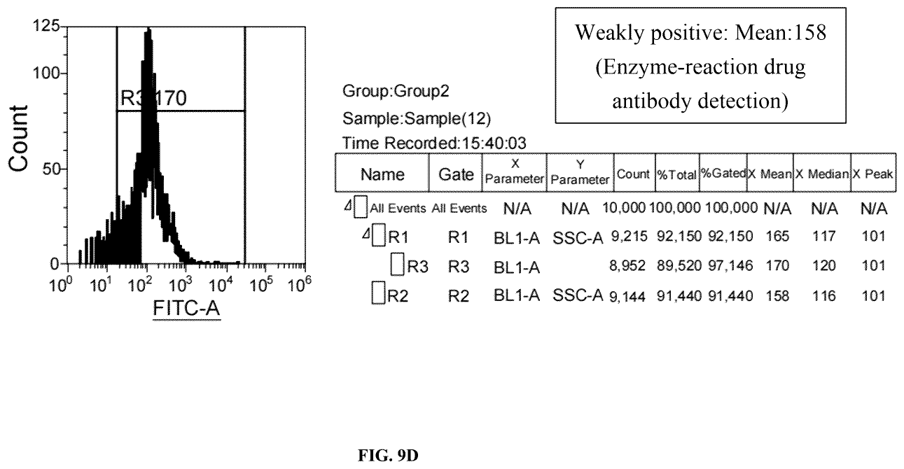

Wu, a patient from the Third Hospital of the City, was tested on the 7th day of ceftriaxone administration in this medical history (2021.12.2-8), and the drug antibody to ceftriaxone was detected (by flow cytometer: Attune WXT). The results were shown in FIGS. 9A-D, the drug antibody to ceftriaxone was weakly positive by enzyme reaction.

### Example 6

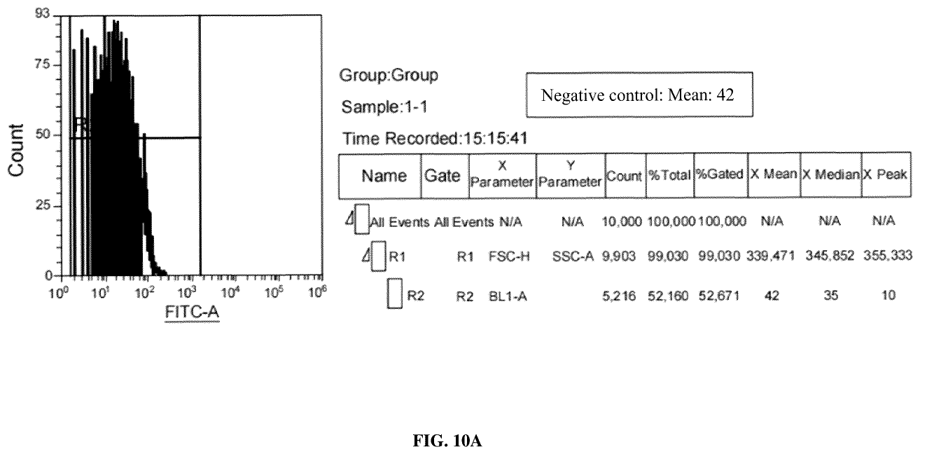

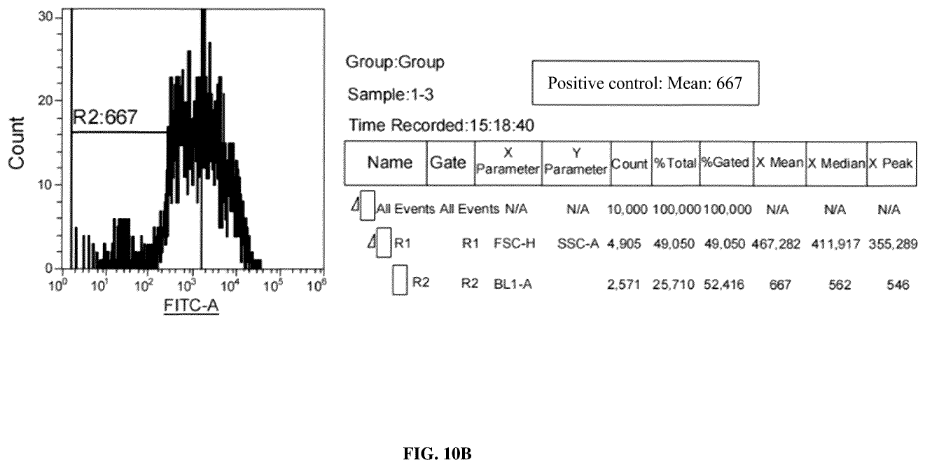

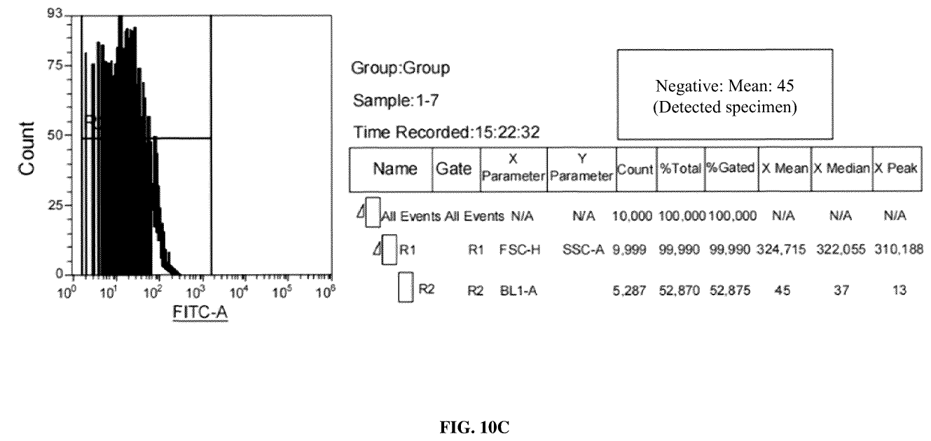

Gao, a patient in the Eighth Affiliated Hospital, Sun Yat-sen University, had no medication history (after the test result was negative, the inventors communicated with the hospital again and learned that the patient began to be treated with piperacillin only after blood was collected and sent for examination in the hospital). The drug antibody to piperacillin was detected (by flow cytometer: Attune WXT). The results were shown in FIGS. 10A-C, the drug antibody to piperacillin was negative.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1. A method for detecting a drug antibody in a specimen, comprising the following steps: preparing at least nine reaction systems; conducting flow cytometry on the at least nine reaction systems sequentially; and determining presence or absence of the drug antibody, a type of the drug antibody, and an affinity of the drug antibody to red blood cells according to results of the flow cytometry; wherein
the nine reaction systems comprise a blank system, a negative control system, a positive control system, a self-control system, a drug-sensitized self-cell control system, a drug-antibody reaction detection system, an enzyme-sensitized self-control system, an enzyme-reaction drug detection system, and an enzyme-reaction drug control system:
the blank system comprises self cells and phosphate-buffered saline (PBS);
the negative control system comprises RhD negative cells, IgG anti-D serum, and the PBS;
the positive control system comprises RhD positive cells, the IgG anti-D serum, and the PBS;
the self-control system comprises the self cells, the PBS, and self plasma;
the drug-sensitized self-cell control system comprises the self cells, the PBS, and a drug liquid;
the drug-antibody reaction detection system comprises drug-sensitized self cells, the drug liquid, and the self plasma;
the enzyme-sensitized self-control system comprises enzyme-sensitized self cells, the PBS, and the self plasma;
the enzyme-reaction drug detection system comprises the enzyme-sensitized self cells, the drug liquid, and the self plasma;
the enzyme-reaction drug control system comprises the enzyme-sensitized self cells, the drug liquid, and the PBS; and
the drug liquid is a drug solution that produces a drug antibody to be tested.

2. The method according to claim 1, wherein the self plasma is derived from a serum specimen or a plasma specimen of a patient with a medication history.

3. The method according to claim 2, wherein the self plasma is a supernatant obtained after conducting centrifugation on the serum specimen or the plasma specimen of the patient with the medication history; and
the centrifugation is conducted at 10,000 rpm for 15 min to 20 min.

4. The method according to claim 1, wherein the self cells are self red blood cells of the patient with the medication history.

5. The method according to claim 1, wherein a preparation method of the drug-sensitized self cells comprises: mixing a normal saline instead of plasma of a patient with self cells of the patient and a drug at a medication concentration of the human body, and conducting incubation to obtain the drug-sensitized self cells.

6. The method according to claim 1, wherein a preparation method of the enzyme-sensitized self cells comprises: mixing self cells of a patient with a papain solution at an equal volume, and conducting incubation to obtain the enzyme-sensitized self cells.

7. The method according to claim 1, wherein the self cells, the drug-sensitized self cells, and the enzyme-sensitized self cells each are configured into a 5% suspension during preparation of respective systems.

8. The method according to claim 7, wherein each 150 μL of the blank system comprises 50 μL of the self cells (5%) and 100 μL of the PBS;
each 300 μL of the negative control system comprises 100 μL of the RhD negative cells (5%), 100 μL of the IgG anti-D serum, and 100 μL of the PBS;
each 300 μL of the positive control system comprises 100 μL of the RhD positive cells (5%), 100 μL of the IgG anti-D serum, and 100 μL of the PBS;
each 300 μL of the self-control system comprises 100 μL of the self cells (5%), 100 μL of the PBS, and 100 μL of the self plasma;
each 300 μL of the drug-sensitized self-cell control system comprises 100 μL of the self cells (5%), 100 μL of the PBS, and 100 μL of the drug liquid;
each 300 μL of the drug-antibody reaction detection system comprises 100 μL of the drug-sensitized self cells (5%), 100 μL of the drug liquid, and 100 μL of the self plasma;
each 300 μL of the enzyme-sensitized self-control system comprises 100 μL of the enzyme-sensitized self cells (5%), 100 μL of the PBS, and 100 μL of the self plasma;
each 300 μL of the enzyme-reaction drug detection system comprises 100 μL of the enzyme-sensitized self cells (5%), 100 μL of the drug liquid, and 100 μL of the self plasma; and
each 300 μL of the enzyme-reaction drug control system comprises 100 μL of the enzyme-sensitized self cells (5%), 100 μL of the drug liquid, and 100 μL of the PBS.

9. The method according to claim 8, further comprising the following steps before the flow cytometry is conducted: conducting incubation on each of the reaction systems at 37° C., mixing with a fluorescently-labeled antibody diluent, and conducting incubation in the dark.

10. The method according to claim 1, wherein a type of an induction drug of the drug antibody comprises a drug-sensitized type, a drug-added type, and an enzyme-treated drug-antibody immune complex type.

11. The method according to claim 5, wherein the self cells, the drug-sensitized self cells, and the enzyme-sensitized self cells each are configured into a 5% suspension during preparation of respective systems.

12. The method according to claim 6, wherein the self cells, the drug-sensitized self cells, and the enzyme-sensitized self cells each are configured into a 5% suspension during preparation of respective systems.

13. The method according to claim 11, wherein each 150 μL of the blank system comprises 50 μL of the self cells and 100 μL of the PBS;
each 300 μL of the negative control system comprises 100 μL of the RhD negative cells, 100 μL of the IgG anti-D serum, and 100 μL of the PBS;
each 300 μL of the positive control system comprises 100 μL of the RhD positive cells, 100 μL of the IgG anti-D serum, and 100 μL of the PBS;
each 300 μL of the self-control system comprises 100 μL of the self cells, 100 μL of the PBS, and 100 μL of the self plasma;
each 300 μL of the drug-sensitized self-cell control system comprises 100 μL of the self cells, 100 μL of the PBS, and 100 μL of the drug liquid;
each 300 μL of the drug-antibody reaction detection system comprises 100 μL of the drug-sensitized self cells (5%), 100 μL of the drug liquid, and 100 μL of the self plasma;
each 300 μL of the enzyme-sensitized self-control system comprises 100 μL of the enzyme-sensitized self cells, 100 μL of the PBS, and 100 μL of the self plasma;
each 300 μL of the enzyme-reaction drug detection system comprises 100 μL of the enzyme-sensitized self cells, 100 μL of the drug liquid, and 100 μL of the self plasma; and
each 300 μL of the enzyme-reaction drug control system comprises 100 μL of the enzyme-sensitized self cells, 100 μL of the drug liquid, and 100 μL of the PBS.

14. The method according to claim 12, wherein each 150 μL of the blank system comprises 50 μL of the self cells and 100 μL of the PBS;
each 300 μL of the negative control system comprises 100 μL of the RhD negative cells, 100 μL of the IgG anti-D serum, and 100 μL of the PBS;
each 300 μL of the positive control system comprises 100 μL of the RhD positive cells, 100 μL of the IgG anti-D serum, and 100 μL of the PBS;
each 300 μL of the self-control system comprises 100 μL of the self cells, 100 μL of the PBS, and 100 μL of the self plasma;
each 300 μL of the drug-sensitized self-cell control system comprises 100 μL of the self cells, 100 μL of the PBS, and 100 μL of the drug liquid;
each 300 μL of the drug-antibody reaction detection system comprises 100 μL of the drug-sensitized self cells (5%), 100 μL of the drug liquid, and 100 μL of the self plasma;
each 300 μL of the enzyme-sensitized self-control system comprises 100 μL of the enzyme-sensitized self cells, 100 μL of the PBS, and 100 μL of the self plasma;
each 300 μL of the enzyme-reaction drug detection system comprises 100 μL of the enzyme-sensitized self cells, 100 μL of the drug liquid, and 100 μL of the self plasma; and
each 300 μL of the enzyme-reaction drug control system comprises 100 μL of the enzyme-sensitized self cells, 100 μL of the drug liquid, and 100 μL of the PBS.

15. The method according to claim 13, further comprising the following steps before the flow cytometry is conducted: conducting incubation on each of the reaction systems at 37° C., mixing with a fluorescently-labeled antibody diluent, and conducting incubation in the dark.

16. The method according to claim 14, further comprising the following steps before the flow cytometry is conducted: conducting incubation on each of the reaction systems at 37° C., mixing with a fluorescently-labeled antibody diluent, and conducting incubation in the dark.
